(19) United States
(12) Patent Application Publication
(10) Pub. No.: US 2026/0124150 A1
(43) Pub. Date: May 7, 2026

(54) USE OF MONOLITHIC ANION EXCHANGE CHROMATOGRAPHY AND LIGHT SCATTERING FOR QUANTIFYING EXTRACELLULAR VESICLES

(71) Applicant: LONZA SALES AG, Basel (CH)

(72) Inventor: Gang Gary HAO, Cambridge, MA (US)

(21) Appl. No.: 18/996,059

(22) PCT Filed: Jul. 13, 2023
(86) PCT No.: PCT/US2023/070140

§ 371 Date: Jan. 17, 2025

Related U.S. Application Data
(60) Provisional application No. 63/390,266, filed on Jul. 18, 2022.

(51) Int. Cl. A61K 9/50; A61K 38/20; B01D 15/36; G01N 30/74; G01N 30/88; G01N 33/543; G01N 33/68

(52) U.S. Cl. CPC A61K 9/5068; A61K 9/5089; A61K 38/208; B01D 15/363; G01N 30/74; G01N 33/543; G01N 33/6869; G01N 2030/8813; G01N 2333/5434

(57) ABSTRACT

The present disclosure relates to methods of detecting and purifying EVs (e.g., exosomes) present within a sample using monolithic anionic exchange chromatography and light scattering. Such methods allow for a more accurate and high-throughput method of quantifying EVs present in a sample. Some aspects of the methods provided herein further comprise analyzing one or more additional properties of EVs.

### FIELD OF DISCLOSURE

The present disclosure relates to methods of quantifying extracellular vesicles (e.g., exosomes) in a sample (e.g., biological sample) using monolithic anion exchange chromatography and light scattering. The methods provided herein can further comprise analyzing one or more properties of the extracellular vesicles present in the sample.

### BACKGROUND OF DISCLOSURE

Extracellular vesicles (e.g., exosomes) are being used in commercial processes, including as therapeutics, and are being produced in industrial quantities. Methods of accurately and rapidly measuring extracellular vesicle presence, purity, concentration and absolute number in complex matrices remain scarce. Current approaches for the detection, isolation and purification of biological extracellular vesicles derived from cell culture or other biological samples requires laborious and time-consuming methods. For example, current ultra-centrifugation protocols are commercially unreproducible, as they produce a heterogeneous mix of extracellular vesicles, other cellular vesicles and macromolecular complexes and can lead to vesicle aggregation. Therefore, novel methods for efficient, low-cost and reliable purification and quantification of such extracellular vesicles are needed.

### SUMMARY OF DISCLOSURE

Provided herein is a method of determining the amount of extracellular vesicle (EV) present in a sample, the method comprising: (i) contacting the sample with a monolithic anion exchange (AEX) chromatography column, and (ii) then measuring a light scattering emission signal from an eluent collected from the AEX chromatography column. Also provided herein is a method of preparing an extracellular vesicle (EV) fraction from a sample, the method comprising determining the amount of EV present in the sample, wherein the determining comprises: (i) contacting the sample with a monolithic anion exchange (AEX) chromatography column, and (ii) then measuring a light scattering emission signal from an eluent collected from the AEX chromatography column.

The present disclosure further provides a method of reducing the amount of impurity present in a sample comprising an extracellular vesicle (EV), the method comprising (i) contacting the sample with a monolithic anion exchange (AEX) chromatography column, and (ii) then measuring a light scattering emission signal from an eluent collected from the AEX chromatography column.

In some aspects, the amount of impurity is reduced by at least about 5%, at least about 10%, at least about 20%, at least about 30%, at least about 40%, at least about 50%, at least about 60%, at least about 70%, at least about 80%, at least about 90%, or at least about 100%, as compared to that of a reference sample (e.g., correspondent eluent collected after contacting the sample to a non-monolithic AEX chromatography column). In some aspects, if the purity of the EV present in the eluent is less than that of a reference value, the method further comprises subjecting the eluent to one or more purification steps. In some aspects, the one or more purification steps comprise a filtration, centrifugation, chromatography, or combinations thereof.

In any of the methods provided herein (e.g., provided above), in some aspects, the monolithic AEX chromatography column comprises a porous surface with a pore size of about 6 μm or greater. In some aspects, the monolithic column comprises a monolithic tertiary amine column.

In any of the methods described herein (e.g., provided above), in some aspects, the eluent is collected after contacting the AEX chromatography column with an elution buffer, wherein the contacting with the elution buffer occurs after (i) (i.e., contacting the sample with the AEX chromatography column). In some aspects, the elution buffer comprises tris, salt, or both. In some aspects, the elution buffer comprises about 50 mM tris, about 2,000 mM NaCl, with a pH of about 7.4. In some aspects, the elution buffer further comprises sodium azide.

In any of the methods described herein (e.g., provided above), in some aspects, the method further comprises contacting the AEX chromatography column with a wash buffer, wherein the contacting with the wash buffer occurs after (i) (i.e., contacting the sample with the AEX chromatography column) and before (ii) (i.e., measuring a light scattering emission signal from the eluent). In some aspects, the wash buffer comprises tris, salt, or both. In some aspects, the wash buffer comprises about 50 mM tris, about 200 mM NaCl with a pH of about 7.4. In some aspects, the wash buffer further comprises sodium azide.

In any of the methods described herein (e.g., provided above), in some aspects, the light scattering emission signal is generated using an excitation wavelength of about 280 nm to about 700 nm. In some aspects, the light scattering emission signal is generated using an excitation wavelength of about 400 nm to about 500 nm. In some aspects, the light scattering emission signal is generated using an excitation wavelength of about 420 nm to about 480 nm. In some aspects, the light scattering emission signal is generated using an excitation wavelength of about 460 nm. In some aspects, the light scattering emission signal is measured at an emission wavelength which is about 0 nm to about 20 nm longer or shorter than the excitation wavelength. In some aspects, the light scattering emission signal is measured at an emission wavelength which is about 10 nm longer or shorter than the excitation wavelength. In some aspects, the light scattering emission signal is measured at an emission wavelength of about 300 nm to about 600 nm. In some aspects, the light scattering emission signal is measured at an emission wavelength of about 400 nm to about 500 nm. In some aspects, the light scattering emission signal is measured at an emission wavelength of about 470 nm. In some aspects, the light scattering emission signal is generated using an excitation wavelength of about 460 nm and measured at an emission wavelength of about 470 nm. In some aspects, the light scattering emission signal is generated using an excitation wavelength of about 280 nm and measured at an emission wavelength of about 350 nm.

In any of the methods described herein (e.g., provided above), in some aspects, the method further comprises analyzing one or more properties of the eluent. In some aspects, the one or more properties comprise an average particle size, a particle count, a polydispersity index, a light scattering intensity, a cholesterol content, a protein content, a lipid content, a payload, or a combination thereof. In some aspects, the one or more properties are measured using an immunoassay selected from an AlphaLISA, dynamic light scattering (DLS), electron microscopy (e.g., transmission electron microscopy or cryogenic electron microscopy) or both.

In some aspects, the average particle size of the eluent is reduced compared to that of a reference sample (e.g., sample prior to the contacting with the AEX chromatography column). In some aspects, the average particle size of the eluent is reduced by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold or more as compared to that of the reference sample. In some aspects, the average particle size of the eluent is between about 20 nm to about 300 nm. In some aspects, the average particle size of the eluent is about 180 nm.

In some aspects, the polydispersity index of the eluent is reduced compared to that of a reference sample (e.g., sample prior to the contacting with the AEX chromatography column). In some aspects, the polydispersity index of the eluent is reduced by at least about 10%, at least about 20%, at least about 30%, at least about 40%, at least about 50%, at least about 60%, at least about 70%, at least about 80%, or at least about 90% or more as compared to that of the reference sample. In some aspects, the polydispersity index of the eluent is between about 0 to about 0.5. In some aspects, the polydispersity index is about 0.13.

In any of the methods described herein (e.g., provided above), in some aspects, the method further comprises subjecting the eluent to one or more purification steps. In some aspects, subjecting the eluent to one or more purification steps occurs after (ii) measuring a light scattering emission signal from the eluent. In some aspects, the one or more purification steps comprise filtration, centrifugation, chromatography, or combinations thereof.

In any of the methods described herein (e.g., provided above), in some aspects, the sample is derived from a cell culture. In some aspects, the cell culture comprises a perfusion cell culture, fed-batch cell culture, or both. In some aspects, the cell culture comprises a mammalian cell. In some aspects, the mammalian cell comprises a human embryonic kidney cell, mesenchymal stem cell, neuronal cell, or a combination thereof.

In any of the methods described herein (e.g., provided above), in some aspects, the EV comprises an exogenous biologically active molecule. In some aspects, the exogenous biologically active molecule comprises a payload, a targeting moiety, or both. In some aspects, the payload comprises a therapeutic molecule, adjuvant, immune modulator, or combinations thereof.

In some aspects, the EV further comprises a scaffold moiety. In some aspects, the scaffold moiety comprises a Scaffold X, a Scaffold Y, or both. In some aspects, the Scaffold X comprises prostaglandin F2 receptor negative regulator (the PTGFRN protein), basigin (the BSG protein), immunoglobulin superfamily member 2 (the IGSF2 protein), immunoglobulin superfamily member 3 (the IGSF3 protein), immunoglobulin superfamily member 8 (the IGSF8 protein), integrin beta-1 (the ITGB1 protein), integrin alpha-4 (the ITGA4 protein), 4F2 cell-surface antigen heavy chain (the SLC3A2 protein), a class of ATP transporter proteins (the ATP1A1, ATP1A2, ATP1A3, ATP1A4, ATP1B3, ATP2B1, ATP2B2, ATP2B3, ATP2B4 proteins), aminopeptidase N (ANPEP; CD13), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin (MFGE8), LAMP2, LAMP2B, or any combination thereof. In some aspects, the Scaffold Y comprises myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or any combination thereof.

In some aspects, the exogenous biologically active molecule is linked to the EV via a scaffold moiety. In some aspects, the exogenous biologically active molecule is linked to the scaffold moiety via a linker. In some aspects, the linker comprises a polypeptide, a non-polypeptide moiety, or both.

In any of the methods described herein (e.g., provided above), in some aspects, the EV comprises an exosome.

Also provided herein is a composition comprising EVs prepared by any of the methods described herein. Also provided herein is a method of treating a disease or condition in a subject in need thereof, comprising administering any of the composition provided herein.

### BRIEF DESCRIPTION OF FIGURES

FIG. 1 provides a schematic of the monolithic anionic exchange quasi-light scattering (mAEX-qLS) quantitation and fractionation method described herein. HEK293 cell culture media were clarified by centrifugation or filtration and injected to mAEX column. The EV particles are resolved from cell debris, proteins and non-EV particles with a short stepwise salt elution. Light scattering detection was enabled on a fluorescence detector to selectively quantitate EV particles and remove interfering co-eluting proteins. The EV peak is further automatically fractioned and assayed for size, lipids and surface payload, etc.

FIGS. 2A and 2B provide chromatograms of harvest media from EV-producing HEK293 cell line. FIG. 2A shows an overlay of chromatograms (Ex280/Em350 nm) for (i) ultracentrifuged harvest medium; (ii) harvest medium; (iii) blank; and (iv) purified EV standard.

FIG. 2B shows an overlay of light scattering chromatograms (Ex460/Em470 nm) for the same groups. Inset: EV and flanking peaks, including a pre-peak and an extended tailing region.

FIG. 3 provides the hydrodynamic radius (Rₙ) (“diameter”) and polydispersity (PDI) of the purified EV standard as measured using Dynamic Light Scattering (DLS).

FIG. 4 provides an overlay of chromatograms (Ex460/Em470 nm) of purified EV standard with and without treatment with proteinase K.

FIGS. 5A and 5B provide the relationship between EV concentration and mAEX peak area and dynamic light scattering intensity, respectively.

FIG. 6 provides the relationship between IL-12 protein concentration measured in the fraction collected after mAEX (x-axis) and the concentration of IL-12-expressing EV particles injected onto the mAEX columns (y-axis). The EV particle concentration was measured by NTA. The IL-12 protein concentration was measured using AlphaLISA.

FIGS. 7A and 7B provide a comparison of IL-12 protein concentration in fraction collected after purification of seven different EV clones (clones 1-7) by OptiPrep (FIG. 7A) or by mAEX (FIG. 7B). The IL-12 protein concentration after OptiPrep purification was measured using Western blot. The IL-12 protein concentration after mAEX purification was measured using alphaLISA. In FIG. 7B, the horizontal line represents the limit of quantitation (LOQ).

FIGS. 8A and 8B provide data relating to the clone selection of HEK293SF cell lines producing native and IL-12 decorated EVs. FIG. 8A provides relative EV particle count from harvest media of native HEK293SF cells. FIG. 8B provide EV particle count and IL-12 expression level from harvest media of IL-12 gene transfected HEK293 SF cell lines. Harvest media of small-scale shake flask culture were centrifuged and the supernatant were analyzed by mAEX for particle concentration. For IL12-EV expressing cell line, the EV peak was automatically fractionated followed by AlphaLISA analysis for IL12 quantitation.

### DETAILED DESCRIPTION OF DISCLOSURE

### I. Definitions

Terms used in the claims and specification are defined as set forth below unless otherwise specified.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “light scattering” refers to scattering and/or reflection of a light source from a focal beam. In some embodiments, the light scattering can be detected at a single angle from the source (e.g., 90 degrees), or can be detected at multiple angles (e.g., in the case of multi-angle light scattering). In some embodiments, the light source is a laser. In some embodiments, the light source is at a wavelength in the ultraviolet spectrum, the visual spectrum, the infrared spectrum, or combinations thereof. In some embodiments, the light scattering is elastic. In some embodiments the light scattering is inelastic. In some preferred embodiments, the light scattering is Rayleigh (elastic) light scattering.

As used herein, the term “excitation wavelength” refers to a wavelength of a light source used to excite the samples. The wavelengths of a light source are controlled by appropriate filters to block or pass specific wavelengths. The excitation wavelength corresponds inversely to the radiation energy of the light source, i.e., longer excitation wavelengths indicate lower radiation energy, while shorter excitation wavelengths indicate higher radiation energy. The term “excitation wavelength” is used interchangeably with “absorbance wavelength” or “absorption wavelength.”

As used herein, the term “emission wavelength” refers to the wavelength of a signal emitted, which is then detected by a detector. Emission signals can be emitted as a result of light scattering after nanoparticles are excited by a light source. In some aspects, the light scattering is inelastic. In some aspects, the light scattering is Rayleigh (elastic) light scattering. In some aspects, the light source can be fluorescence, and the emission wavelength is a wavelength of fluorescence. In some aspects, the fluorescence are intrinsic fluorescence. The term “intrinsic fluorescence” refers to a fluorescence naturally occurring after excitation. Such intrinsic fluorescence can occur when molecules such as aromatic amino acids, neurotransmitters, porphyrins, and green fluorescent protein are excited. In contrast, extrinsic fluorescence is emitted from synthetic dyes or modified biochemical molecules that are added to a sample. In some aspects, the detector for fluorescence signal can be a UV/Vis detector. In some aspects, the detector can be a fluorescence detector. In some aspects, the detector can be a multi-wavelength detector.

As used herein, the term “separation” or “fractionation” refers to a process in which a certain quantity of a sample is divided into a number of smaller quantities (fraction) in which the compositions vary. In some aspects, the sample is a mixture. In some aspects, the mixture is a suspension. Fractions are collected based on differences in specific property of individual components. The difference properties on which the separation process is based can be chemical or physical including such properties as chemical reactivity, solubility, molecular size, electrical charge and change-of-phase temperatures such as boiling and freezing points. Separation or fractionation techniques can be broadly classified into processes of mechanical separation and separation by diffusion. Mechanical separation techniques can be based on particle size, density and electrical or magnetic mobility. Separation by diffusion includes chromatographic separation, extraction and fractionation. In some aspects, the fractions are nanoparticles. Broad groupings of such methods and techniques include fractionation proper, general separation, analytical separation and purification. Examples of separation or fractionation can be, but not limited to, dephlegmation, fractional distillation, fractional freezing, fractional melting, isotope fractionation, solvent or clean fractionation, thermal diffusion, centrifugation, ultracentrifugation, gaseous diffusion, chromatography, bioassay-guided fractionation, geochemical fractionation, and purification. In some aspects, the separation or fractionation takes place in column chromatography by a difference in affinity between the stationary phase of the column and the mobile phase of the sample. In some aspects, the column chromatography is size exclusion chromatography (SEC). SEC is a chromatographic method in which molecules in solution are separated by their size, and in some cases molecular weight. It is usually applied to large molecules or macromolecular complexes such as proteins and polymers. The chromatography column can be packed with fine, porous beads which are composed of dextran polymers (SEPHADEX®), agarose (SEPHAROSE®), or polyacrylamide. The pore sizes of these beads are used to estimate the dimensions of macromolecules. In some aspects, the SEC comprises one or more elution steps.

As used herein, the term “batch release assay”, “batch release test”, or “lot release assay” refers to the measurement methods described herein that can be used in the lot or batch release assays that are subjected to Good Manufacturing Practice (GMP) for biologic drug or to GMP for Medicinal Products for Human and Veterinary Use. The purpose of testing and controlling batch release is to ensure that the batch has been manufactured and checked in accordance with the principles and guidelines of GMP, and the relevant records are readily identifiable for future inspection. The release assays can also provide the deviation measurements for the manufacturing process and/or the analytical control methods. The deviation can be assessed in accordance with a quality risk management process using an appropriate approach under the GMP guidelines. The suite of tests (including specifications and details of laboratory methodologies where appropriate) is agreed between the pharmaceutical manufacturer in consultation with regulators during the application for marketing approval. The specific tests vary widely between product types, their mechanisms of action and manufacturing processes. However, a laboratory will typically analyze the physical characteristics of batch samples (for tablets, this could be their color, shape, solubility, etc.) along with a host of tests on the active ingredients to ensure that their concentrations (and any degradation products) are within regulatory tolerance and the manufacturer's own tolerance range (usually 2-5%). Finally, the samples will undergo microbiological and chemical scrutiny to verify the product contains no hazardous materials (for example remnants of the manufacturing process). In some aspects, the physical characteristics of extracellular vesicles (EVs) and nanoparticles manufactured can be measured during one or more release assays. In some aspects, the light scattering at various excitation and emission wavelengths, the intrinsic fluorescence, and the UV absorbance of the EVs and nanoparticles in a batch are measured and recorded to detect and/or quantify the sizes, purity, and concentration of the EVs and nanoparticles in the production. In some aspects, the light scattering signal is measured at the excitation wavelength of 460 nm and the emission wavelength at 470 nm. In some aspects, the intrinsic fluorescence is measured at the excitation wavelength of 280 nm and the emission wavelength at 350 nm. In some aspects, the UV absorbance is measured at the wavelength of 280 nm. In some aspects, other measurements to assess the identity and concentration of the nanoparticles (e.g., extracellular vesicles) described herein can be included in the release assays.

As used herein, “nanoparticle” refers to a small physical entity that is between 1 nm and 1,000 nm in size as measured by its longest axis (e.g., its diameter if spherical). Nanoparticles may be produced by cells or may be synthetic, or a combination or mixture thereof. Nanoparticles may be monodisperse, polydisperse, homogeneous, or heterogeneous. Nanoparticles may exist in a complex mixture of various excipients, salts, biological material, synthetic material, matrices, gels, or other formulations known in the art, whether natural or synthetic. In a nonlimiting example, a virus-like particle (VLP) is a species of nanoparticle.

As used herein, the term “extracellular vesicle” or “EV” refers to a cell-derived vesicle comprising a membrane that encloses an internal space. Extracellular vesicles comprise all membrane-bound vesicles (e.g., exosomes or nanovesicle) that have a smaller diameter than the cell from which they are derived. In some aspects, extracellular vesicles range in diameter from 20 nm to 1000 nm, and may comprise various macromolecular cargo either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles may be derived from a living or dead organism, explanted tissues or organs, prokaryotic or eukaryotic cells, and/or cultured cells. In some aspects, the extracellular vesicles are produced by cells that express one or more transgene products. An extracellular vesicle is a species of nanoparticle.

As used herein, the term “nanovesicle” refers to a cell-derived small (between 20-250 nm in diameter, e.g., between 30-150 nm) vesicle comprising a membrane that encloses an internal space, and which is generated from a cell (e.g., producer cell) by direct or indirect manipulation such that the nanovesicle would not be produced by the producer cell without the manipulation. Appropriate manipulations of the producer cell include but are not limited to serial extrusion, treatment with alkaline solutions, sonication, or combinations thereof. Generally, production of nanovesicles does not result in the destruction of the producer cell. In some aspects, populations of nanovesicles are substantially free of vesicles that are derived from producer cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The nanovesicle, once it is derived from a producer cell according to the manipulation, may be isolated from the producer cell based on its size, density, biochemical parameters, or a combination thereof. A nanovesicle is a species of extracellular vesicle.

As used herein, the term “exosome” refers to a cell-derived small (between 20-300 nm in diameter, e.g., between 40-200 nm) vesicle comprising a membrane that encloses an internal space (i.e., lumen), and in some aspects, can be generated from a cell (e.g., producer cell) by direct plasma membrane budding or by fusion of the late endosome with the plasma membrane. In certain aspects, an exosome comprises a scaffold moiety. Generally, production of exosomes does not result in the destruction of the producer cell. The exosome comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. As described infra, the exosome can be derived from a producer cell, and isolated from the producer cell based on its size, density, biochemical parameters, or a combination thereof. An exosome is a species of extracellular vesicle. In some aspects, the EVs, e.g., exosomes, of the present disclosure are produced by cells that express one or more transgene products.

As used herein, the terms “parent cell” or “producer cell” include any cell from which an extracellular vesicle may be isolated. The terms also encompass a cell that shares a protein, lipid, sugar, or nucleic acid component of the extracellular vesicle. For example, a “parent cell” or “producer cell” may include a cell which serves as a source for the extracellular vesicle membrane.

As used herein, the term “synthetic nanoparticle” is a small (between 1-1,000 nm as measured by its longest axis) object or structure that is not produced from living cells. Synthetic nanoparticles may contain biological macromolecules such as lipids, proteins, nucleic acids and/or carbohydrates, but cannot be produced by living cells. A liposome, lipid nanoparticle, detergents, other polymeric structures, a synthetic bead (e.g., polystyrene bead, quantum dot, or metal bead) and a DNA nanostructure are all species of synthetic nanoparticle. In some embodiments, the synthetic nanoparticle is spherical or near-spherical.

As used herein, the terms “purify,” “purified,” and “purifying” or “isolate,” “isolated,” or “isolating” or “enrich,” “enriched” or “enriching” are used interchangeably and refer to the state of a population (e.g., a plurality of known or unknown amount and/or concentration) of desired extracellular vesicles, that have undergone one or more processes of purification, e.g., a selection or an enrichment of the desired extracellular vesicles composition, or alternatively a removal or reduction of residual biological products as described herein. In some embodiments, a purified extracellular vesicles composition has no detectable undesired activity or, alternatively, the level or amount of the undesired activity is at or below an acceptable level or amount. In other embodiments, a purified extracellular vesicle composition has an amount and/or concentration of desired extracellular vesicles at or above an acceptable amount and/or concentration. In other embodiments, the purified extracellular vesicle composition is enriched as compared to the starting material (e.g., biological material collected from tissue, bodily fluid, or cell preparations) from which the composition is obtained. This enrichment may be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. The term “purity” as used herein, refers to the amount and/or concentration of desired extracellular vesicles or nanoparticles over the total amount of the samples. As used herein, the term “contaminant” or “impurity” refers to the fractions (e.g., proteinaceous species, cellular debris, raptured extracellular vesicles or nanoparticles, microvesicles, apoptotic debris, or other impurities) other than the desired extracellular vesicles or nanoparticles in the samples.

Abbreviations used in this application include the following: Size-exclusion chromatography (SEC), Anion Exchange Chromatography (AEX), Two-dimensional liquid chromatography (2D-LC), Nanoparticle tracking analysis (NTA), Resistive pulse sensing (RPS), extracellular vesicles (EV or EVs), Phosphate Buffered Saline (PBS) and Fluorescent Activated Cell Sorting (FACS).

### II. Methods of the Disclosure

Described herein are methods for the detection and quantification of extracellular vesicles from complex matrices such as samples. In some aspects, the disclosure provides a method of determining the amount of extracellular vesicle (EV) present in a sample, the method comprising: (i) contacting the sample with a monolithic anion exchange (AEX) chromatography column, and (ii) then measuring a light scattering emission signal from an eluent collected from the AEX chromatography column. In some aspects, the disclosure provides a method of preparing an extracellular vesicle (EV) fraction from a sample, the method comprising determining the amount of EV present in the sample, wherein the determining comprises: (i) contacting the sample with a monolithic anion exchange (AEX) chromatography column, and (ii) then measuring a light scattering emission signal from an eluent collected from the AEX chromatography column. In some aspects, the disclosure provides a method of reducing the amount of impurity present in a sample comprising an extracellular vesicle (EV), the method comprising (i) contacting the sample with a monolithic anion exchange (AEX) chromatography column, and (ii) then measuring a light scattering emission signal from an eluent collected from the AEX chromatography column.

In some aspects, the amount of impurity is reduced by at least about 5%, at least about 10%, at least about 20%, at least about 30%, at least about 40%, at least about 50%, at least about 60%, at least about 70%, at least about 80%, at least about 90%, or at least about 100%, as compared to that of a reference sample (e.g., correspondent eluent collected after contacting the sample to a non-monolithic AEX chromatography column). In some aspects, if the purity of the EV present in the eluent is less than that of a reference value, the method further comprises subjecting the eluent to one or more purification steps.

In some aspects, the methods of the present disclose further comprise a one or more purification steps comprising a filtration, centrifugation, chromatography, or combinations thereof.

In some aspects, the monolithic AEX chromatography column comprises a porous surface with a pore size of at least about 4 μm, at least about 5 μm, at least about 6 μm, at least about 7 μm, or at least about 8 μm. In some aspects, the mAEX column comprises a porous surface with a pore size of about 4 μm-about 12 μm, about 4 μm-about 11 μm, about 4 μm-about 10 μm, about 4 μm-about 9 μm, about 4 μm-about 8 μm, about 4 μm-about 7 μm, about 4 μm-about 6 μm, about 4 μm-about 5 μm, about 5 μm μm-about 12 μm, about 5 μm-about 11 μm, about 5 μm-about 10 μm, about 5 μm-about 9 μm, about 5 μm-about 8 μm, about 5 μm-about 7 μm, about 5 μm-about 6 μm, about 6 μm-about 11 μm, about 6 μm-about 10 μm, about 6 μm-about 9 μm, about 6 μm-about 8 μm, about 6 μm-about 7 μm, about 7 μm-about 11 μm, about 7 μm-about 10 μm, about 7 μm-about 9 μm, about 7 μm-about 8 μm, about 8 μm-about 11 μm, about 8 μm-about 10 μm, or about 8 μm-about 9 μm.

In some aspects, the monolithic column comprises a monolithic tertiary amine column.

In some aspects, the elution buffer comprises tris, salt, or both. In some aspects, the elution buffer comprises about 50 mM tris, about 2,000 mM NaCl, with a pH of about 7.4. In some aspects, the elution buffer further comprises sodium azide.

In some aspects, the method further comprises contacting the AEX chromatography column with a wash buffer, wherein the contacting with the wash buffer occurs after (i) (i.e., contacting the sample with the AEX chromatography column) and before (ii) (i.e., measuring a light scattering emission signal from the eluent). In some aspects, the wash buffer comprises tris, salt, or both. In some aspects, the wash buffer comprises about 50 mM tris, about 200 mM NaCl with a pH of about 7.4. In some aspects, the wash buffer further comprises sodium azide.

In some aspects, the light scattering emission signal is generated using an excitation wavelength of about 280 nm to about 700 nm. In some aspects, the light source has an excitation wavelength ranging from about 300 nm to about 700 nm, from about 320 nm to about 700 nm, from about 340 nm to about 700 nm, from about 360 nm to about 700 nm, from about 380 nm to about 700 nm, from about 400 nm to about 700 nm, from about 420 nm to about 700 nm, from about 440 nm to about 700 nm, from about 460 nm to about 700 nm, from about 300 nm to about 660 nm, from about 320 nm to about 660 nm, from about 340 nm to about 660 nm, from about 360 nm to about 660 nm, from about 380 nm to about 660 nm, from about 400 nm to about 660 nm, from about 420 nm to about 660 nm, from about 440 nm to about 660 nm, from about 460 nm to about 660 nm, from about 300 nm to about 640 nm, from about 320 nm to about 640 nm, from about 340 nm to about 640 nm, from about 360 nm to about 640 nm, from about 380 nm to about 640 nm, from about 400 nm to about 640 nm, from about 420 nm to about 640 nm, from about 440 nm to about 640 nm, from about 460 nm to about 640 nm, from about 400 nm to about 600 nm, from about 400 nm to about 500 nm, from about 450 nm to about 500 nm, from about 420 nm to about 520 nm, or from about 440 nm to about 540 nm. In some aspects, the light source has an excitation wavelength ranging from about 400 nm to about 500 nm.

In some aspects, the light scattering emission signal has an emission wavelength equal to or longer than the excitation wavelength. In some aspects, the emission wavelength is less than about 20 nm, less than about 19 nm, less than about 18 nm, less than about 17 nm, less than about 16 nm, less than about 15 nm, less than about 14 nm, less than about 13 nm, less than about 12 nm, less than about 11 nm, less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm longer than the excitation wavelength.

In some aspects, the emission wavelength is about 1 nm to about 20 nm, about 1 nm to about 19 nm, about 1 nm to about 18 nm, about 1 nm to about 17 nm, about 1 nm to about 16 nm, about 1 nm to about 15 nm, about 1 nm to about 14 nm, about 1 nm to about 13 nm, about 1 nm to about 12 nm, about 1 nm to about 11 nm, about 1 nm to about 10 nm, about 1 nm to about 9 nm, about 2 nm to about 20 nm, about 2 nm to about 19 nm, about 2 nm to about 18 nm, about 2 nm to about 17 nm, about 2 nm to about 16 nm, about 2 nm to about 15 nm, about 2 nm to about 14 nm, about 2 nm to about 13 nm, about 2 nm to about 12 nm, about 2 nm to about 11 nm, about 2 nm to about 10 nm, about 2 nm to about 9 nm, about 3 nm to about 20 nm, about 3 nm to about 19 nm, about 3 nm to about 18 nm, about 3 nm to about 17 nm, about 3 nm to about 16 nm, about 3 nm to about 15 nm, about 3 nm to about 14 nm, about 3 nm to about 13 nm, about 3 nm to about 12 nm, about 3 nm to about 11 nm, about 3 nm to about 10 nm, about 3 nm to about 9 nm, about 4 nm to about 20 nm, about 4 nm to about 19 nm, about 4 nm to about 18 nm, about 4 nm to about 17 nm, about 4 nm to about 16 nm, about 4 nm to about 15 nm, about 4 nm to about 14 nm, about 4 nm to about 13 nm, about 4 nm to about 12 nm, about 4 nm to about 11 nm, about 4 nm to about 10 nm, about 4 nm to about 9 nm, about 5 nm to about 20 nm, about 5 nm to about 19 nm, about 5 nm to about 18 nm, about 5 nm to about 17 nm, about 5 nm to about 16 nm, about 5 nm to about 15 nm, about 5 nm to about 14 nm, about 5 nm to about 13 nm, about 5 nm to about 12 nm, about 5 nm to about 11 nm, about 5 nm to about 10 nm, about 5 nm to about 9 nm, about 6 nm to about 20 nm, about 6 nm to about 19 nm, about 6 nm to about 18 nm, about 6 nm to about 17 nm, about 6 nm to about 16 nm, about 6 nm to about 15 nm, about 6 nm to about 14 nm, about 6 nm to about 13 nm, about 6 nm to about 12 nm, about 6 nm to about 11 nm, about 6 nm to about 10 nm, about 6 nm to about 9 nm, about 7 nm to about 20 nm, about 7 nm to about 19 nm, about 7 nm to about 18 nm, about 7 nm to about 17 nm, about 7 nm to about 16 nm, about 7 nm to about 15 nm, about 7 nm to about 14 nm, about 7 nm to about 13 nm, about 7 nm to about 12 nm, about 7 nm to about 11 nm, about 7 nm to about 10 nm, about 7 nm to about 9 nm, about 8 nm to about 20 nm, about 8 nm to about 19 nm, about 8 nm to about 18 nm, about 8 nm to about 17 nm, about 8 nm to about 16 nm, about 8 nm to about 15 nm, about 8 nm to about 14 nm, about 8 nm to about 13 nm, about 8 nm to about 12 nm, about 8 nm to about 11 nm, about 8 nm to about 10 nm, about 8 nm to about 9 nm, about 9 nm to about 20 nm, about 9 nm to about 19 nm, about 9 nm to about 18 nm, about 9 nm to about 17 nm, about 9 nm to about 16 nm, about 9 nm to about 15 nm, about 9 nm to about 14 nm, about 9 nm to about 13 nm, about 9 nm to about 12 nm, about 9 nm to about 11 nm, about 9 nm to about 10 nm, about 10 nm to about 20 nm, about 10 nm to about 19 nm, about 10 nm to about 18 nm, about 10 nm to about 17 nm, about 10 nm to about 16 nm, about 10 nm to about 15 nm, about 10 nm to about 14 nm, about 10 nm to about 13 nm, about 10 nm to about 12 nm, or about 10 nm to about 11 nm longer than the excitation wavelength. In some aspects, the difference between the emission and excitation wavelengths ranges from about 5 nm to about 20 nm. In some aspects, the difference between the emission and excitation wavelengths ranges from about 10 nm to about 20 nm.

In some aspects, the difference between the emission and excitation wavelengths is selected from a group comprising 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, and 20 nm.

In some aspects, the light source has an excitation wavelength at about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, or about 700 nm. In some aspects, the emission wavelength is between 300 nm and about 320 nm, between 310 nm and about 330 nm, between 320 nm and about 340 nm, between 330 nm and about 350 nm, between 340 nm and about 360 nm, between 350 nm and about 370 nm, between 360 nm and about 380 nm, between 370 nm and about 390 nm between 380 nm and about 400 nm, between 390 nm and about 410 nm, between 400 nm and about 420 nm, between 410 nm and about 430 nm, between 420 nm and about 440 nm, between 430 nm and about 450 nm, between 440 nm and about 460 nm, between 450 nm and about 470 nm, between 460 nm and about 480 nm, between 470 nm and about 490 nm, between 480 nm and about 500 nm, between 490 nm and about 510 nm, between 500 nm and about 520 nm, between 510 nm and about 530 nm, between 520 nm and about 540 nm, between 530 nm and about 550 nm, between 540 nm and about 560 nm, between 550 nm and about 570 nm, between 560 nm and about 580 nm, between 570 nm and about 590 nm, between 580 nm and about 600 nm, between 590 nm and about 610 nm, between 600 nm and about 620 nm, between 610 nm and about 630 nm, between 620 nm and about 640 nm, between 630 nm and about 650 nm, between 640 nm and about 660 nm, between 650 nm and about 670 nm, between 660 nm and about 680 nm, between 670 nm and about 690 nm, between 680 nm and about 700 nm, between 690 nm and about 710 nm, or between 700 nm and about 720 nm, respectively.

In some aspects, the light source has an excitation wavelength at about 450 nm to about 470 nm. In some aspects, the excitation wavelength is about 460 nm and the emission wavelength is about 460 nm to about 480 nm. In some aspects, the excitation wavelength is about 460 nm and the emission wavelength is about 460 nm, about 465 nm, about 470 nm, about 475 nm, or about 480 nm. In some aspects, the excitation wavelength is about 450 nm and the emission wavelength is about 450 nm to about 470 nm. In some aspects, the excitation wavelength is about 450 nm and the emission wavelength is about 450 nm, about 455 nm, about 460 nm, about 465 nm, or about 470 nm. In some aspects, the excitation wavelength is about 470 nm and the emission wavelength is about 470 nm to about 490 nm. In some aspects, the excitation wavelength is about 470 nm and the emission wavelength is about 470 nm, about 475 nm, about 480 nm, about 485 nm, or about 490 nm.

In some aspects, the excitation wavelength is about 550 nm to about 560 nm and the emission wavelength is about 550 nm to about 580 nm and wherein the excitation wavelength is the same or shorter than the emission wavelength. In some aspects, the excitation wavelength is about 556 nm and the emission wavelength is about 556 nm, about 560 nm, about 565 nm, about 570 nm, or about 573 nm. In some aspects, the light scattering emission signal is measured by a UV/Vis detector or a fluorescence detector.

In some aspects, the light scattering emission signal is generated using an excitation wavelength of about 460 nm and measured at an emission wavelength of about 470 nm. In some aspects, the light scattering emission signal is generated using an excitation wavelength of about 280 nm and measured at an emission wavelength of about 350 nm.

In some aspects, the methods of the present disclosure further comprises analyzing one or more properties of the eluent. In some aspects, the one or more properties comprise an average particle size, a particle count, a polydispersity index, a light scattering intensity, a cholesterol content, a protein content, a lipid content, a payload, or a combination thereof. In some aspects, the one or more properties are measured using an immunoassay selected from an AlphaLISA, dynamic light scattering (DLS), electron microscopy (e.g., transmission electron microscopy or cryogenic electron microscopy) or both.

In some aspects, the average particle size of the eluent is reduced compared to that of a reference sample (e.g., sample prior to the contacting with the AEX chromatography column). In some aspects, the average particle size of the eluent is reduced by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold or more as compared to that of the reference sample. In some aspects, the average particle size of the eluent is between about 20 nm to about 300 nm. In some aspects, the average particle size of the eluent is about 180 nm.

In some aspects, the polydispersity index of the eluent is reduced compared to that of a reference sample (e.g., sample prior to the contacting with the AEX chromatography column). In some aspects, the polydispersity index of the eluent is reduced by at least about 10%, at least about 20%, at least about 30%, at least about 40%, at least about 50%, at least about 60%, at least about 70%, at least about 80%, or at least about 90% or more as compared to that of the reference sample. In some aspects, the polydispersity index of the eluent is between about 0 to about 0.5. In some aspects, the polydispersity index is about 0.13.

In some aspects, the method of the disclosure further comprises subjecting the eluent to one or more purification steps. In some aspects, subjecting the eluent to one or more purification steps occurs after (ii) measuring a light scattering emission signal from the eluent.

In some aspects, the one or more purification steps of the present methods comprise filtration, centrifugation, chromatography, or combinations thereof.

Samples for the present methods can include, but are not limited to, raw cell culture harvest, clarified cell culture medium, enriched extracellular vesicle preparations, partially purified extracellular vesicle preparations (e.g., by a single two-hour ultracentrifugation step), or highly purified extracellular vesicle preparations (e.g., extracellular vesicle preparations additionally purified using a density gradient medium (e.g., sucrose density gradient medium or medium comprising an iodixanol solution (Sigma-Aldrich))).

The parent cell can be cultured. Cultured parent cells can be scaled up from bench-top scale to bioreactor scale. For example, the parent cells are cultured until they reach saturation density, e.g., 1×10⁵, 1×10⁶, 1×10⁷, or greater than 1×10⁷ complexes per ml. Optionally, upon reaching saturation density, the parent cells can be transferred to a larger volume of fresh medium. The parent cells may be cultured in a bioreactor, such as, e.g., a Wave-type bioreactor, a stirred-tank bioreactor. Various configurations of bioreactors are known in the art and a suitable configuration may be chosen as desired. Configurations suitable for culturing and/or expanding populations of parent cells can easily be determined by one of skill in the art without undue experimentation. The bioreactor can be oxygenated. The bioreactor may optionally contain one or more impellers, a recycle stream, a media inlet stream, and control components to regulate the influx of media and nutrients or to regulate the outflux of media, nutrients, and waste products. In some aspects, the culturing methods comprise a perfusion culture method.

Extracellular vesicles can be made synthetically or isolated from a biological system such as a cell or an organism. The EVs can contain biological macromolecules such as lipids, proteins, nucleic acids and/or carbohydrates. Methods of making EVs are well-known in the art. For example, nanoparticles such as liposomes, lipid nanoparticles, detergents, beads, and other polymeric structures, can be produced by extrusion, emulsion, sonication, mixing, self-assembly, lithography, or crystallization.

With respect to purification or enrichment of extracellular vesicles, it is contemplated that all known manners of purification of extracellular vesicles are deemed suitable for use herein. For example, physical properties of extracellular vesicles may be employed to separate them from a medium or other source material, including separation on the basis of electrical charge (e.g., electrophoretic separation, ion-exchange chromatography), size (e.g., filtration, size-exclusion chromatography, molecular sieving, etc.), density (e.g., regular or gradient centrifugation), Svedberg constant (e.g., sedimentation with or without external force, etc.). For ion-exchange chromatography, any suitable methods known in the art may be used including, but not limited to, anion-exchange chromatography, and strong-anion exchange chromatography. For density gradient centrifugation, any appropriate density gradient medium used in the art may be used, including, but not limited to, sucrose density gradient medium and mediums comprising, iodixanol solution, colloidal silica, inorganic salts, polyhydric alcohols, polysaccharides, poly(vinyl alcohol), iohexol and nonionic iodinated media. Purification of the extracellular vesicles may be performed by manually loading columns or other devices, or may be automated using devices such an autosampler.

Alternatively, or additionally, isolation can be based on one or more biological properties, and include methods that can employ surface markers (e.g., precipitation, reversible binding to solid phase, FACS separation, separation using magnetic surfaces, specific ligand binding, immunoprecipitation or other antibody-mediated separation techniques, non-specific ligand binding such as annexin V, etc.). In yet further contemplated methods, the extracellular vesicles can also be fused using chemical and/or physical methods, including PEG-induced fusion and/or ultrasonic fusion.

In some aspects, enrichment of extracellular vesicles can be done in a general and non-selective manner (e.g., methods comprising serial centrifugation), and can be performed by aggregation where the extracellular vesicles are interlinked with an interlinking composition (e.g., annexin V, fibrin, or an antibody or fragment thereof against at least one of a tetraspanin, ICAM-1, CD86, CD63, PTGFRN, BASP1, or any combination thereof). Non-limiting examples of the interlinking composition are found in U.S. Pat. No. 10,195,290 B1, issued Feb. 5, 2019 and US Publication No. 2019/0151456 A1 published May 23, 2019. Alternatively, enrichment of extracellular vesicles can be done in a more specific and selective manner (e.g., using tissue or cell specific surface markers). For example, specific surface markers can be used in immunoprecipitation, FACS sorting, and/or bead-bound ligands for magnetic separation, etc.

In some aspects, size exclusion chromatography can be utilized to enrich the extracellular vesicles. Size exclusion chromatography techniques are known in the art. Exemplary, non-limiting techniques are provided herein. In some aspects, a void volume fraction is isolated and comprises extracellular vesicles of interest. Further, in some aspects, the extracellular vesicles can be further isolated after chromatographic separation by centrifugation techniques (of one or more chromatography fractions), as is generally known in the art. In some aspects, for example, density gradient centrifugation can be utilized to further enrich the extracellular vesicles. Still further, in some aspects, it can be desirable to further separate the parent-cell derived extracellular vesicles from extracellular vesicles of other origin. For example, the parent cell derived extracellular vesicles can be separated from non-parent cell-derived extracellular vesicles by immunosorbent capture using an antigen antibody specific for the parent cell. For example, anti-IL-12 or anti-PTGFRN antibodies can be used.

In some aspects, the isolation of extracellular vesicles can involve combinations of methods that include, but are not limited to, differential centrifugation, size-based membrane filtration, concentration and/or rate zonal centrifugation, and further characterized using methods that include, but are not limited to, electron microscopy, flow cytometry and/or Western blotting.

Extracellular vesicles can be extracted from the supernatant of parent cells and demonstrate membrane and internal protein, lipid, and nucleic acid compositions that enable their efficient delivery to and interaction with recipient cells. Extracellular vesicles can be derived from parent cells that may include, but are not limited to, reticulocytes, erythrocytes, megakaryocytes, platelets, neutrophils, tumor cells, connective tissue cells, neural cells and stem cells. Suitable sources of extracellular vesicles include but are not limited to, cells isolated from subjects from patient-derived hematopoietic or erythroid progenitor cells, immortalized cell lines, or cells derived from induced pluripotent stem cells, optionally cultured and differentiated. Cell culture protocols can vary according to compositions of nutrients, growth factors, starting cell lines, culture period, and morphological traits by which the resulting cells are characterized. In some embodiments, the samples comprising extracellular vesicles are derived from a plurality of donor cell types (e.g., 2, 3, 4, 5, 6, 7, 8, 9, 10, 50, 100, 500, 1000, 5000, or 10000 donor cell types) and are combined or pooled. Pooling may occur by mixing cell populations prior to extracellular vesicles extraction or by mixing isolated extracellular vesicles compositions from subsets of donor cell types. Parent cells may be irradiated or otherwise treated to affect the production rate and/or composition pattern of secreted extracellular vesicles prior to isolation.

In certain aspects, the extracellular vesicles may be derived from cell lines that are differentiated, proliferated and cultured in-vitro. This enables controllable and reproducible compositions of extracellular vesicles that are not subject to constraints on isolation and purification of the requisite parent cell type.

In certain aspects, the samples comprising the extracellular vesicles are obtained from raw cell harvest and the light scattering signature is determined. In certain aspects, the raw cell harvest is clarified for larger cells and cellular debris prior to determination of the light scattering signature. In certain aspects, the samples comprising the extracellular vesicles are further purified using any of the above mentioned methods for enrichment of the extracellular vesicles prior to determination of the light scattering signature of the samples.

In certain aspects, the methods comprise fractionating the sample prior to determination of the light scattering signature. In certain aspects, the method comprises the steps of loading the extracellular vesicle preparation on a size exclusion chromatography (SEC) column (e.g., a sepharose resin SEC column). In certain aspects, the methods comprise the steps of loading the extracellular vesicle preparation on an ion exchange chromatography column. In some aspects, the methods comprise the steps of loading the extracellular vesicle preparation on a strong anion exchange chromatography column.

In some aspects, the cell culture comprises a mammalian cell. In some aspects, the mammalian cell comprises a human embryonic kidney cell, mesenchymal stem cell, neuronal cell, or a combination thereof.

In some aspects, the EV comprises an exogenous biologically active molecule. In some aspects, the exogenous biologically active molecule comprises a payload, a targeting moiety, or both. In some aspects, the payload comprises a therapeutic molecule, adjuvant, immune modulator, or combinations thereof.

In some aspects, the EV further comprises a scaffold moiety. In some aspects, the scaffold moiety comprises a Scaffold X, a Scaffold Y, or both. In some aspects, the Scaffold X comprises prostaglandin F2 receptor negative regulator (the PTGFRN protein), basigin (the BSG protein), immunoglobulin superfamily member 2 (the IGSF2 protein), immunoglobulin superfamily member 3 (the IGSF3 protein), immunoglobulin superfamily member 8 (the IGSF8 protein), integrin beta-1 (the ITGB1 protein), integrin alpha-4 (the ITGA4 protein), 4F2 cell-surface antigen heavy chain (the SLC3A2 protein), a class of ATP transporter proteins (the ATP1A1, ATP1A2, ATP1A3, ATP1A4, ATP1B3, ATP2B1, ATP2B2, ATP2B3, ATP2B4 proteins), aminopeptidase N (ANPEP; CD13), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin (MFGE8), LAMP2, LAMP2B, or any combination thereof. In some aspects, the Scaffold Y comprises myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or any combination thereof.

In some aspects, the exogenous biologically active molecule is linked to the EV via a scaffold moiety. In some aspects, the exogenous biologically active molecule is linked to the scaffold moiety via a linker. In some aspects, the linker comprises a polypeptide, a non-polypeptide moiety, or both.

In some aspects, the disclosure comprises a composition comprising the EV prepared by the method of the present disclosure. In some aspects, the disclosure comprises a method of treating a disease or condition in a subject in need thereof, comprising administering the composition of the present disclosure to the subject.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Materials and Methods

The following examples use one or more of the materials and methods described below:

### HEK293 Cell Culture

The HEK293SF cell line was transfected with a mammalian expression vector expressing interleukin-12 (IL12) using Transporter 5 PEI (Polysciences) and stably expressing cells were selected with puromycin (Invivogen). Once the transfected cells recovered from selection, they were single cell cloned by plating at a seeding density of 0.5 cells/well in 384 well plates. Monoclonality was confirmed by plate imaging at day 0 with a Cellavista 4 (Synentec). Viable clones were expanded through a series of multi well plates to small scale shake flasks. Shake flask fed batch cultures were harvested on day 7 with the media clarified by centrifugation prior to analysis. The same culture condition was used for clone screening of the native HEK293SF cell line.

mAEX and Fractionation

Anion exchange chromatography (AEX) was performed on a Waters Acquity UPLC system equipped with fluorescence detector and fraction manager. 50 μL of EV samples were injected to a custom-built AEX column (CIMac™ quaternary amine monolith 0.1 mL, 6 um pore size) from BIA Separations (Ajdov ̌s ̌cina, Slovenia). The column was washed with mobile phase A (50 mM Tris, 200 mM NaCl, pH 7.4) for 2 min and EVs were eluted with 60% mobile phase B (50 mM Tris, 2 M NaCl, pH 7.4) over three min at a flow rate of 1 mL/min. Column eluent was monitored at Ex 280 nm/Em350 nm for tryptophan residues and at Ex460 nm/Em470 nm for particle light scattering. The emission wavelength at 460 nm was chosen for the lack of interference from proteins, carbohydrates or lipids and a 10 nm shift was used as emission to satisfy the requirement of the Empower software for fluorescence detection. The particles will scatter the emission light and the side-scattered lights are detected on the diagonal axle at the detector. Automatic peak fractionation is carried out on the Acquity Fraction Manager based on predefined retention time.

### AlphaLISA Immunoassay

The EV samples were incubated for an hour with a solution of an anti-IL-12 (p70) conjugated acceptor bead (10 ug/mL) and biotinylated anti-IL-12 (p40) (1 ug/mL) in a AlphaPlate. Following incubation, a solution of streptavidin donor beads (80 ug/mL) were added and incubated in the dark for 1 hour. A microplate reader (BMG Clariostar) was used to excite the donor beads at 680 nm and read the emission wavelength of the acceptor beads between 515-520 nm. The IL12 concentration in EV samples was calculated from standard curve generated from recombinant IL12 (R&D biosystems).

### Dynamic Light Scattering (DLS)

Dynamic light scattering using the Wyatt DynaPro Plate Reader III instrument. NIST polystyrene size standards (80 nm, 150 nm, and 250 nm) were used for calibration. 200 μL of collected AEX samples were added to UV-transparent 96 well plate and the DLS data was acquired in triplicate wells at 25° C. The raw data was fitted with Dynamics software to obtain size distribution of hydrodynamic radius.

### Example 2: mAEX-qLS Method Development and Qualification

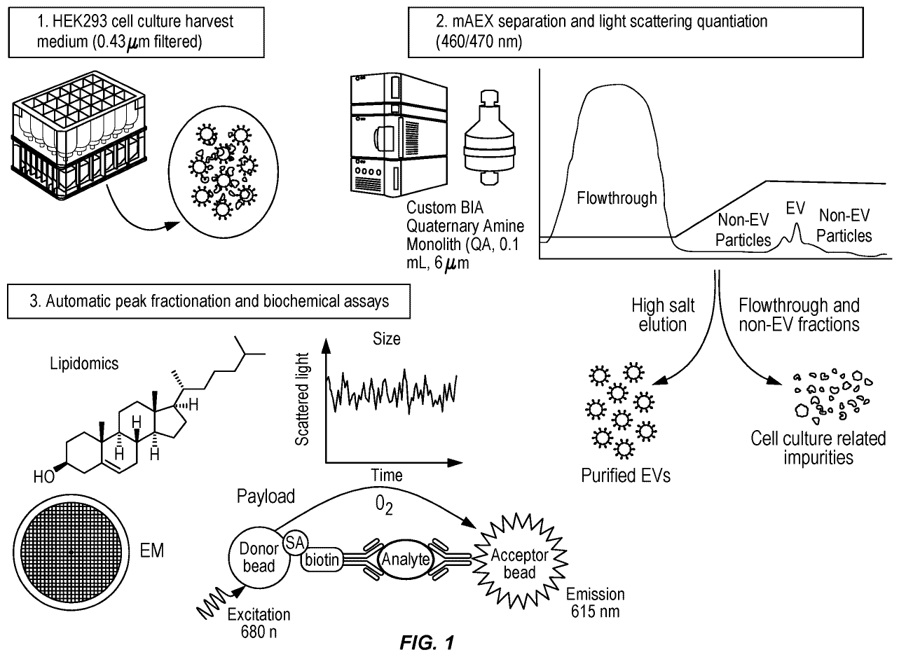

The workflow for the mAEX-qLS method described herein is illustrated in FIG. 1. Briefly, clarified cell culture harvest (e.g., HEK293 cell) is injected onto mAEX column to resolve EV from interfering matrix (e.g., contaminants) and the concentration of EV is quantified by light scattering enabled on conventional fluorescence detector. The EV peak is automatically fraction collected followed by orthogonal characterization.

### Method Development

In developing the above method, the suitability of different chromatographic matrices was first assessed. Size exclusion chromatography (SEC) was found to inadequately resolve EV from other similarly sized particles and suffered rapid column deterioration from repetitive injection of the crude samples. Anion exchange (AEX) separation was explored to leverage the negative surface charge of EVs. Strong anion exchange column (ProPac SAX, ThermoFisher) provided efficient separation but was also prone to column fouling.

To reduce the above issues, a custom ordered monolithic tertiary amine anion exchange (mAEX) column was selected. For instance, the selected mAEX column had large pore size (6 μm) enabling rapid mass transfer and elongated column durability. Additionally, the column was short (0.1 ml column) allowing for faster resolution of the EVs from the other contaminants present in the sample. A short (8 min) stepwise salt elution was applied to resolve the mixture and the eluent was monitored by intrinsic protein fluorescence (Ex 280/Em 350 nm) to maximize detection sensitivity.

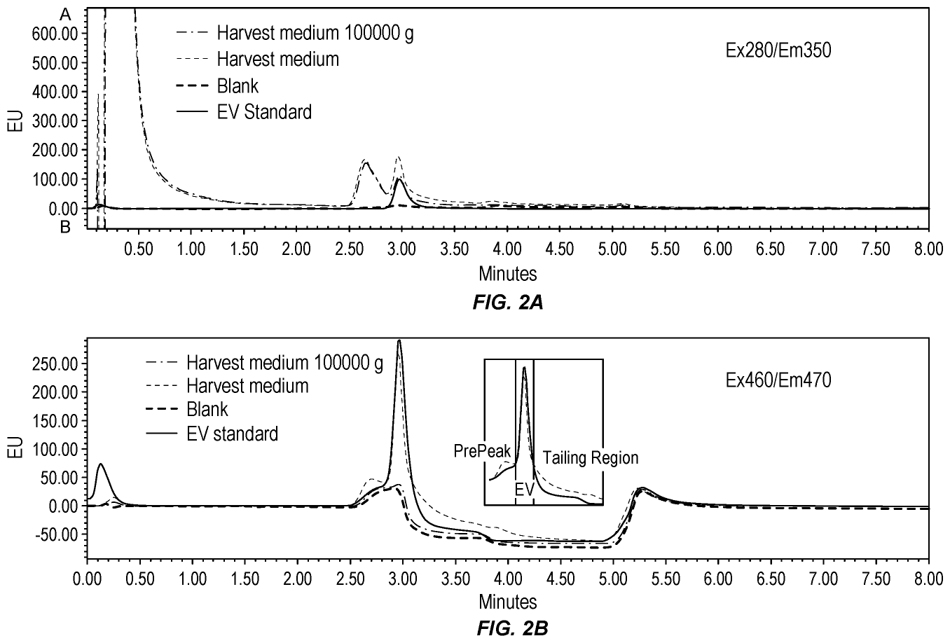

As shown in FIG. 2A, most proteinaceous species flowed through the column at the initial salt concentration of 200 mM NaCl. The EV species eluted around 3 min based on the retention time alignment with purified EV standard.

Next, the presence of non-particle species co-eluting under the EV peak was investigated by ultracentrifugation of the culture medium at 10,000 g. Again, as shown in FIG. 2A, nearly half of the EV peak area remained from the ultracentrifuged sample, indicating significant presence of proteinaceous species which would result in the overestimation of the EV concentration.

To circumvent this problem, light scattering detection was implemented to minimize the interference from co-eluting species, leveraging the exponentially increased scattering intensity of the EV particles compared to proteins. A quasi-light scattering (qLS) detection (Ex460 nm/Em 470 nm) was implemented on conventional fluorescence detector (FLR) to avoid the need for dedicated multiangle light scattering detection system and to facilitate robust data acquisition and integration within the Water Empower software.

As shown in FIG. 2B, the 3 min EV peak appeared more prominent in the harvest medium sample and completely disappeared following ultracentrifugation, corroborating the preferential response of qLS towards particulates. A distinct peak preceding the EV (˜2.5 min) and an extended tailing region were observed from the harvest medium sample (FIG. 2B inset). These species were absent in the EV standard, indicating the efficient removal of these impurities through the purification process described herein. The negative baseline after 3 min was an artifact associated with change in salt concentration as evidenced from the blank injection. Based on these results, qLS signal was used for EV quantitation and the particle number was obtained from comparing peak area to standard curve generated from purified EV standard.

### Method Qualification

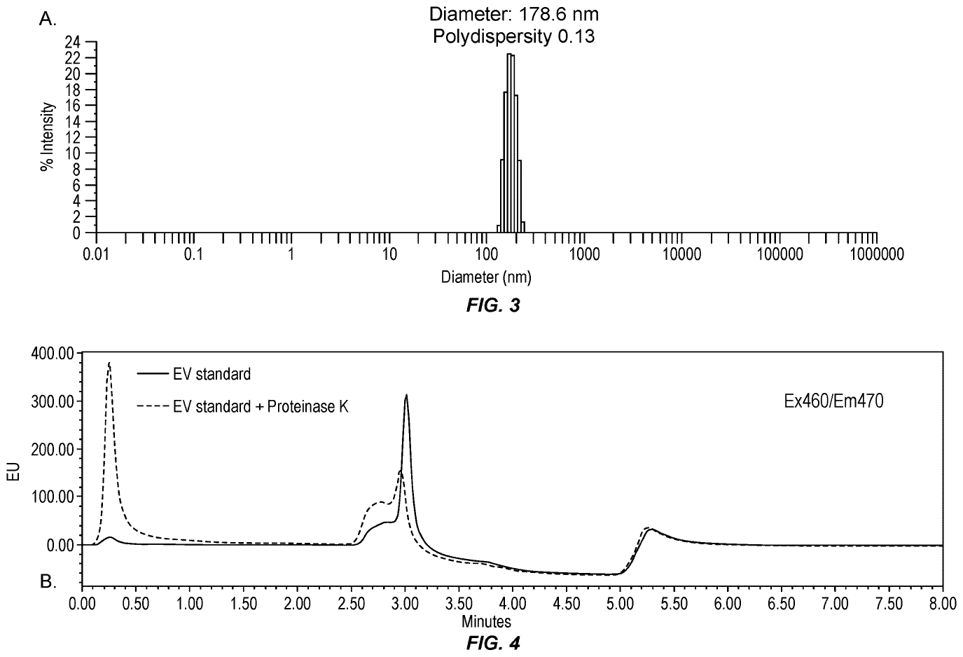

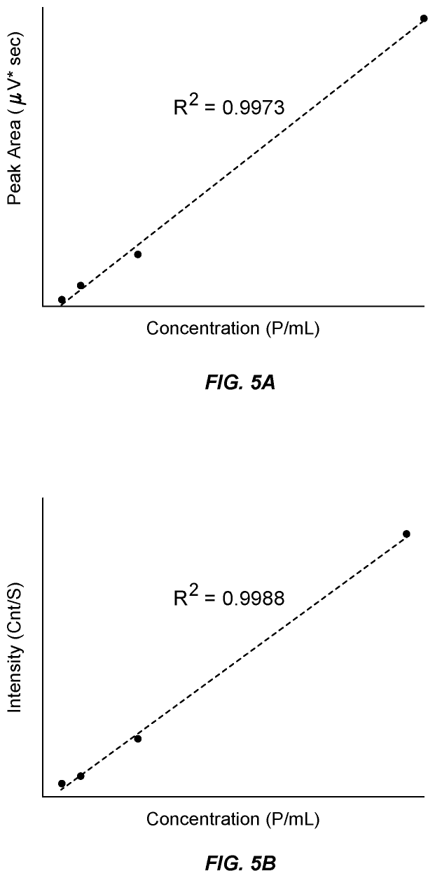

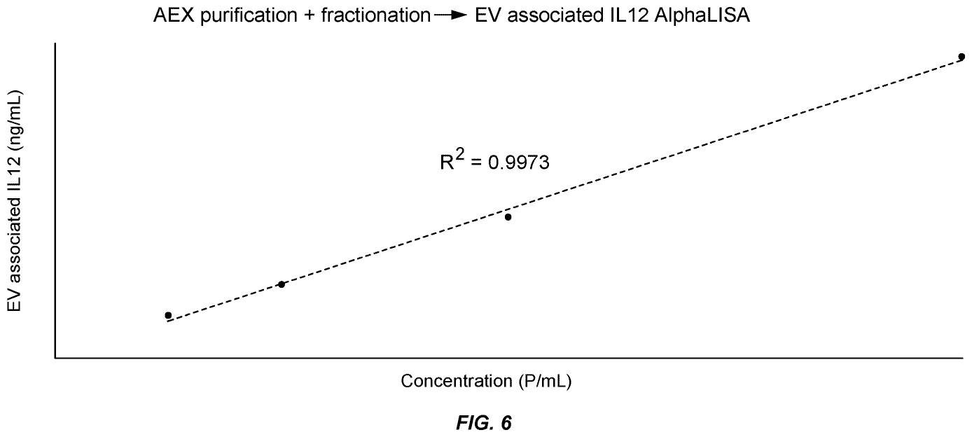

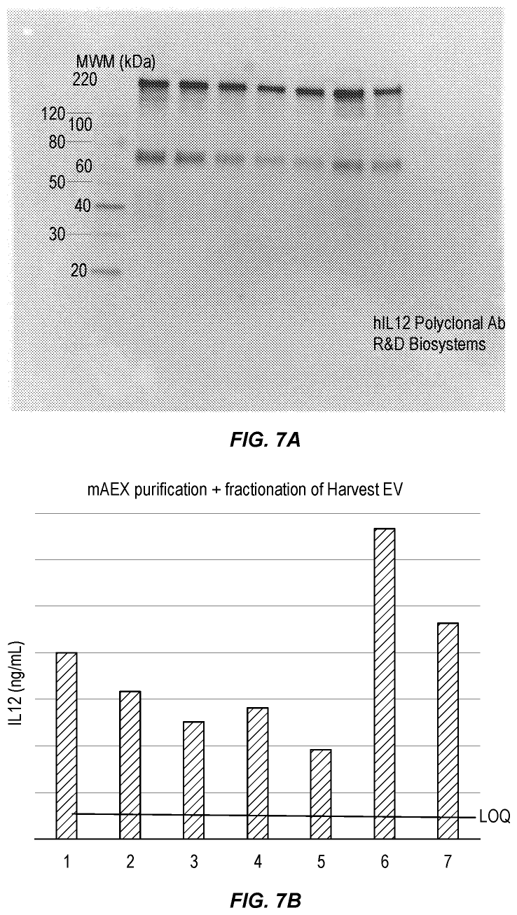

Next, to confirm that the methods described above could be useful in quantifying EV concentrations, the identities of the EV and surrounding peaks depicted in FIG. 2B inset were investigated. The main EV peak was fraction collected for particle size and polydispersity measurement on dynamic light scattering (DLS). As shown in FIG. 3, the hydrodynamic radius of the fractionated species was centered around 180 nm, consistent with the expected range for HEK293 cell derived EVs. The polydispersity index of 0.13 indicated the relative homogeneity of the main peak.

The structural identity of the pre-EV peak was investigated by mild proteinase K digestion under non-denaturing condition (PBS buffer at 37° C. for 1 hour). Interestingly, as shown in FIG. 4, the digestion resulted in increased pre-peak intensity with concomitant shift of the main peak towards earlier elution time. This result suggested the pre-peak as a form of protein naïve EV species. Preliminary results revealed the presence of histone as well as extracellular matrix proteins, that might form complex with DNA and the EV, respectively.

The methods described herein were further assessed for specificity, reproducibility, linearity, accuracy and robustness (stability) and the results are summarized in Table 1.

TABLE 1
Method Qualification Summary

| | | | | | Stability |
|---|---|---|---|---|---|
| | | | | | (column |
| | | Precision | | | pressure |
| | | (Peak Area | Accuracy | Linear Range | and half |
| Criteria | Specificity | RSD %) | (Recovery %) | (P/mL) | peak width) |
| Results | Lack of | Intraday 5% | 2.5E10 96% | 1.25E10-10E10 | Injection 1 |
| | interfering | Interday 5% | 5.0E10 100% | R² = 0.9973 | 1832 psi |
| | peaks on blank | | 7.5E10 103% | | 0.08 min |
| | injection | | | | Injection 200 |
| | Retention time | | | | 1835 psi |
| | consistent with | | | | 0.08 min |
| | purified EV | | | | |
| | standard | | | | |

The specificity was established from the lack of interfering peaks in the blank injection and the retention time alignment with the EV standard. Precision was assessed from six injections on each of two different days and less than <5% RSD in peak area was achieved. Accuracy was evaluated from spike recovery of sample injected across three concentration levels. Greater than 95% recovery of the peak area was obtained. The linearity was established as from 1.25 E10 to 1E11 particle/mL based on serial dilution of EV standard. The stability of the method was evaluated from comparison of column pressure and half width of the 3 min EV peak between the first injection and two hundredth injection. No noticeable increase in column pressure and decrease in peak resolution was observed following 200 injection of crude media, demonstrating the stability of the monolithic column.

### Application to Clone Selection

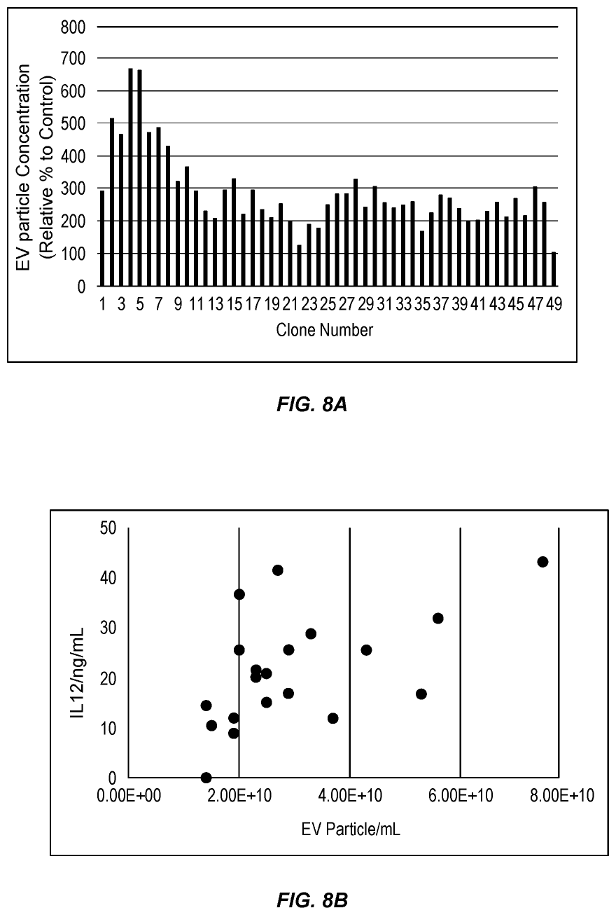

The mAEX platform was applied to clone selection of HEK293SF cell line producing both native and surface engineered EVs. The harvest media on day seven from forty-nine native HEK293SF clones were analyzed for EV concentration and the results are summarized in FIG. 8A. A broad range (more than seven folds) of productivity was found across the native cell line. As mentioned above, the LC based method provided the advantage of peak fractionation to enable profiling of other attributes. The HEK293SF cells stably transfected to express interleukin-12 (IL-12) on EV surface were screened. The anti-tumor bioactivities of the IL-12 decorated EV are mediated by the lipid nanoparticle and the IL-12 and thus the quantification of both moieties is required as criteria for clone selection. The EV peak from the twenty samples were automatically collected and assayed for IL-12 expression level by AlphaLISA and the results are shown in FIG. 8B. As shown, certain clones exhibited three- and five-folds increase over the original cell line in IL-12 and particle yield, respectively. IL-12 was also present on non-EV species, including cell membrane debris and protein aggregates. Without removing these species by mAEX, the AlphaLISA would yield erroneously higher IL-12 concentrations and be unrepresentative of EV-associated IL-12. In addition to IL-12 quantitation, other plate-based techniques have also been coupled to the fractionation to gain insight to the product characteristics, e.g., hydrodynamic radius and polydispersity by dynamic light scattering (DLS) and cholesterol content by Amplex Red assay.

Collectively, the above results demonstrate that the methods provided herein have sufficient throughput and sensitivity to directly measure the fraction collected samples, and thus provide a much superior and high-throughput method of quantifying EVs in a sample (e.g., cell culture harvest). The current run time of under 10 minutes can support 192 samples within 24 hours.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

What is claimed is:

1. A method of determining the amount of extracellular vesicle (EV) present in a sample, the method comprising: (i) contacting the sample with a monolithic anion exchange (AEX) chromatography column, and (ii) then measuring a light scattering emission signal from an eluent collected from the AEX chromatography column.

2. A method of preparing an extracellular vesicle (EV) fraction from a sample, the method comprising: (i) contacting the sample with a monolithic anion exchange (AEX) chromatography column, (ii) collecting an eluent from the monolithic AEX chromatography column, and (iii) then measuring a light scattering emission signal from the eluent to prepare the EV fraction.

3. A method of reducing the amount of impurity present in a sample comprising an extracellular vesicle (EV), the method comprising (i) contacting the sample with a monolithic anion exchange (AEX) chromatography column, (ii) collecting an eluent from the monolithic AEX chromatography column, and (iii) then measuring a light scattering emission signal from the eluent wherein the amount of impurity is reduced in the sample.

4. The method of claim 3, wherein the amount of impurity is reduced by at least about 5%, at least about 10%, at least about 20%, at least about 30%, at least about 40%, at least about 50%, at least about 60%, at least about 70%, at least about 80%, at least about 90%, or at least about 100%, as compared to that of a reference sample (e.g., correspondent eluent collected after contacting the sample to a non-monolithic AEX chromatography column).

5. The method of claim 3 or 4, wherein if the purity of the EV present in the eluent is less than that of a reference value, the method further comprises subjecting the eluent to one or more purification steps.

6. The method of claim 5, wherein the one or more purification steps comprise a filtration, centrifugation, chromatography, or combinations thereof.

7. The method of any one of claims 1 to 6, wherein the monolithic AEX chromatography column comprises a porous surface with a pore size of about 6 μm or greater.

8. The method of any one of claims 1 to 7, wherein the monolithic column comprises a monolithic tertiary amine column.

9. The method of any one of claims 1 to 8, wherein the eluent is collected after contacting the AEX chromatography column with an elution buffer, wherein the contacting with the elution buffer occurs after (i) (i.e., contacting the sample with the AEX chromatography column).

10. The method of claim 9, wherein the elution buffer comprises tris, salt, or both.

11. The method of claim 10, wherein the elution buffer comprises about 50 mM tris, about 2,000 mM NaCl, with a pH of about 7.4.

12. The method of claim 10 or 11, wherein the elution buffer further comprises sodium azide.

13. The method of any one of claims 1 to 12, further comprising contacting the AEX chromatography column with a wash buffer, wherein the contacting with the wash buffer occurs after (i) (i.e., contacting the sample with the AEX chromatography column) and before (ii) (i.e., measuring a light scattering emission signal from the eluent).

14. The method of claim 13, wherein the wash buffer comprises tris, salt, or both.

15. The method of claim 14, wherein the wash buffer comprises about 50 mM tris, about 200 mM NaCl with a pH of about 7.4.

16. The method of claim 14 or 15, wherein the wash buffer further comprises sodium azide.

17. The method of any one of claims 1 to 16, wherein the light scattering emission signal is generated using an excitation wavelength of about 280 nm to about 700 nm.

18. The method of claim 17, wherein the light scattering emission signal is generated using an excitation wavelength of about 400 nm to about 500 nm.

19. The method of claim 18, wherein the light scattering emission signal is generated using an excitation wavelength of about 420 nm to about 480 nm.

20. The method of claim 19, wherein the light scattering emission signal is generated using an excitation wavelength of about 460 nm.

21. The method of any one of claims 1 to 20, wherein the light scattering emission signal is measured at an emission wavelength which is about 0 nm to about 20 nm longer or shorter than the excitation wavelength.

22. The method of claim 21, wherein the light scattering emission signal is measured at an emission wavelength which is about 10 nm longer or shorter than the excitation wavelength.

23. The method of any one of claims 1 to 22, wherein the light scattering emission signal is measured at an emission wavelength of about 300 nm to about 600 nm.

24. The method of claim 23, wherein the light scattering emission signal is measured at an emission wavelength of about 400 nm to about 500 nm.

25. The method of claim 24, wherein the light scattering emission signal is measured at an emission wavelength of about 470 nm.

26. The method of any one of claims 1 to 16, wherein the light scattering emission signal is generated using an excitation wavelength of about 460 nm and measured at an emission wavelength of about 470 nm.

27. The method of any one of claims 1 to 16, wherein the light scattering emission signal is generated using an excitation wavelength of about 280 nm and measured at an emission wavelength of about 350 nm.

28. The method of any one of claims 1 to 27, which further comprises analyzing one or more properties of the eluent.

29. The method of claim 28, wherein the one or more properties comprise an average particle size, a particle count, a polydispersity index, a light scattering intensity, a cholesterol content, a protein content, a lipid content, a payload content, or a combination thereof.

30. The method of claim 28 or 29, wherein the one or more properties are measured using an immunoassay selected from an AlphaLISA, dynamic light scattering (DLS), electron microscopy (e.g., transmission electron microscopy or cryogenic electron microscopy), or combinations thereof.

31. The method of claim 30, wherein the payload content comprises the level of a cytokine present in the eluent.

32. The method of claim 31, wherein the cytokine comprises IL-12.

33. The method of claim 32, wherein the level of IL-12 is measured using an IL-12-specific AlphaLISA.

34. The method of any one of claims 29 to 33, wherein the average particle size of the eluent is reduced compared to that of a reference sample (e.g., sample prior to the contacting with the AEX chromatography column).

35. The method of claim 34, wherein the average particle size of the eluent is reduced by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold or more as compared to that of the reference sample.

36. The method of any one of claim 34 or 35, wherein the average particle size of the eluent is between about 20 nm to about 300 nm.

37. The method of claim 36, wherein the average particle size of the eluent is about 180 nm.

38. The method of any one of claims 29 to 37, wherein the polydispersity index of the eluent is reduced compared to that of a reference sample (e.g., sample prior to the contacting with the AEX chromatography column).

39. The method of claim 38, wherein the polydispersity index of the eluent is reduced by at least about 10%, at least about 20%, at least about 30%, at least about 40%, at least about 50%, at least about 60%, at least about 70%, at least about 80%, or at least about 90% or more as compared to that of the reference sample.

40. The method of claim 38 or 39, wherein the polydispersity index of the eluent is between about 0 to about 0.5.

41. The method of claim 40, wherein the polydispersity index is about 0.13.

42. The method of any one of claims 1, 2, and 6 to 41, which further comprises subjecting the eluent to one or more purification steps.

43. The method of claim 42, wherein subjecting the eluent to one or more purification steps occurs after (ii) measuring a light scattering emission signal from the eluent.

44. The method of claim 42 or 43, wherein the one or more purification steps comprise filtration, centrifugation, chromatography, or combinations thereof.

45. The method of any one of claims 1 to 44, wherein the sample is derived from a cell culture.

46. The method of claim 45, wherein the cell culture comprises a perfusion cell culture, fed-batch cell culture, or both.

47. The method of claim 45 or 46, wherein the cell culture comprises a mammalian cell.

48. The method of claim 47, wherein the mammalian cell comprises a human embryonic kidney cell, mesenchymal stem cell, neuronal cell, or a combination thereof.

49. The method of any one of claims 1 to 48, wherein the EV comprises an exogenous biologically active molecule.

50. The method of claim 49, wherein the exogenous biologically active molecule comprises a payload, a targeting moiety, or both.

51. The method of claim 50, wherein the payload comprises a therapeutic molecule, adjuvant, immune modulator, or combinations thereof.

52. The method of claim 50 or 51, wherein the payload comprises a cytokine.

53. The method of claim 52, wherein the cytokine comprises IL-12.

54. The method of any one of claims 49 to 53, wherein the EV further comprises a scaffold moiety.

55. The method of claim 54, wherein the scaffold moiety comprises a Scaffold X, a Scaffold Y, or both.

56. The method of claim 55, wherein the Scaffold X comprises prostaglandin F2 receptor negative regulator (the PTGFRN protein), basigin (the BSG protein), immunoglobulin superfamily member 2 (the IGSF2 protein), immunoglobulin superfamily member 3 (the IGSF3 protein), immunoglobulin superfamily member 8 (the IGSF8 protein), integrin beta-1 (the ITGB1 protein), integrin alpha-4 (the ITGA4 protein), 4F2 cell-surface antigen heavy chain (the SLC3A2 protein), a class of ATP transporter proteins (the ATP1A1, ATP1A2, ATP1A3, ATP1A4, ATP1B3, ATP2B1, ATP2B2, ATP2B3, ATP2B4 proteins), aminopeptidase N (ANPEP; CD13), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin (MFGE8), LAMP2, LAMP2B, or any combination thereof.

57. The method of claim 55 or 56, wherein the Scaffold Y comprises myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or any combination thereof.

58. The method of any one of claims 55 to 57, wherein the exogenous biologically active molecule is linked to the EV via a scaffold moiety.

59. The method of claim 58, wherein the exogenous biologically active molecule is linked to the scaffold moiety via a linker.

60. The method of claim 59, wherein the linker comprises a polypeptide, a non-polypeptide moiety, or both.

61. The method of any one of claims 1 to 60, wherein the EV comprises an exosome.

62. A composition comprising the EV prepared by the method of any one of claims 1 to

63. A method of treating a disease or condition in a subject in need thereof, comprising administering the composition of claim 62 to the subject.
